(19) United States
(12) Patent Application Publication
(10) Pub. No.: US 2013/0345533 A1
(43) Pub. Date: Dec. 26, 2013

(54) SENSOR FOR SCANNING VITAL TISSUE

(71) Applicants: Holger Jungmann, Gelsenkirchen (DE); Michael Schietzel, Herdecke (DE)

(72) Inventors: Holger Jungmann, Gelsenkirchen (DE); Michael Schietzel, Herdecke (DE)

(21) Appl. No.: 13/884,021

(22) PCT Filed: Nov. 9, 2011
(86) PCT No.: PCT/EP11/05636

§ 371 Date: Sep. 17, 2013

(30) Foreign Application Priority Data
Nov. 9, 2010 (DE) 102010-050-666.4

(51) Int. Cl. A61B 5/00; A61B 5/1455

(52) U.S. Cl. CPC A61B 5/6826; A61B 5/1455
USPC 600/344

(57) ABSTRACT

The invention relates to an application device for a sensor head, which can be used for example as a component of a measuring arrangement for obtaining measurement signals from living tissue in order to determine the presence, concentration, or material composition of bodily fluids and substances possibly only temporarily vessel-bound. The aim of the invention is to create solutions by means of which it is possible to take non-invasive measurements, in particular spectrometric measurements, in a manner that is advantageous in terms of measurement and ergonomics, in the region of the fingers of a user, in particular a patient. Said aim is achieved according to the invention by an application device for applying a sensor head to a finger of a user, comprising an outer housing, an insert body, which is made of a soft material and sits in the outer housing, wherein the insert body forms a finger-receiving cavern, wherein the outer housing has a rear shell section, a first clamping leg, and a second clamping leg, wherein the two clamping legs can be variably positioned relative to the rear shell section in such a way that the insert body can be deformed in a defined manner between the rear shell section and the two clamping legs.

The invention concerns a holder for a sensor head that can be used for example as a part of a measuring device for collecting measuring signals, from living tissue, for determining the presence, concentration, or composition of body liquids as well as of maybe only temporarily vascular-bound substances.

Movable spectrometers are known, by which an analysis of temporarily vascular-bound substances can be done, by applying a sensor head device coupled with the spectrometer to a fingertip of a user, especially a patient, so this spectrometer can each time record the spectrum from reflected light emerging from the fingertip tissue. The spectrum recorded in this way allows various substances present in the examined tissue area to be detected. These spectrometers can be structured like classic spectrometers, in case of which a splitting of the incident light is done by optical means and the intensity of the split light is measured by associating it to wavelength. For avoiding movable parts the spectrometer can be formed in such a way that the light split according to its wavelength is deflected to a CCD array that analyzes its intensity.

DE 4242232 [U.S. Pat. No. 5,687,721] describes a sensor device that analyzes at a patient's fingertip the light emitted by the tissue.

The object of the invention is to create solutions by which it is possible, at a user's, especially a patient's, finger, to perform noninvasive measurements, especially spectrometric measurements, in a metrologically and ergonomically advantageous way.

This object is achieved according to the invention by a holder for applying a sensor head at a user's finger, comprising:
an outer housing,
an insert manufactured from an elastically compressible material and in the outer housing, the insert forming a finger stall,
the outer housing including a back shell, a first holding leg and a second holding leg, the two holding legs being in such a way variably positionable with respect to the back shell that the insert between the back shell and the two holding legs is deformable in a defined way, especially radially compressible.

It is thus advantageously possible to create a holder that is distinguished by high wearing comfort and is precisely adaptable to the size of the finger to be recorded.

According to a particularly preferred embodiment of the invention the insert is exposed at a holding gap between the back shell and the respective holding legs. The insert radially compressed by the respective holding leg can spread radially into and, if need be, through this holding gap. The holding gap in this respect forms an opening, into which the insert can spread from inside.

Moreover according to a particular aspect of the present invention a leg gap through which the insert again is partially exposed is also formed between the two holding legs.

Also the insert can extend and spread in this leg gap radially to some degree.

This leg gap can form an opening extending longitudinally of the finger stall and through which the insert can spread from inside.

According to a particularly preferred embodiment of the invention, the back shell and the holding legs are adjustable relative to one another by an adjusting mechanism. The adjusting mechanism is preferably configured in such a way that the back shell and the holding legs are displaceable relative to one another along an adjusting axis that is substantially orthogonal to the longitudinal axis of the finger stall. This adjusting mechanism preferably includes a guide as well as a set screw. The set screw assembly can steplessly vary a spacing of the holding leg from the back shell. The set screw can be integrated into the holder in such a way that it can be adjusted without tools. Preferably a cap is provided to secure the set screw and cover it in the selected position.

The adjusting mechanism can be formed in such a way that different holding sizes can be set by it for the area adjacent the fingertip of the finger stall and for the area forming the insertion opening of the finger stall. In this way it is possible to adapt the holder advantageously to differently shaped fingers.

The two holding legs are preferably united in a leg structure that is formed in one piece as an integral part. This leg structure can include a base where the two legs are connected to each other. This base section preferably also houses part of the adjusting mechanism.

The leg gap between the holding legs in the insert preferably forms an application opening for receiving a sensor head. The insert or the holding legs can grip the sensor head in the holder. Preferably the structure for securing the sensor head is configured in such a way that contact pressure of the sensor head on the tissue area to be examined is limited, preferably that it corresponds relatively precisely to the pressure exerted by the insert.

The locating opening of the sensor head provided at the leg gap is especially suitable for measurements at the mesial finger surfaces.

For carrying out measurements at the back of the finger or for transmitted light measurements advantageously also at the back section an opening is provided for receiving another sensor head.

Preferably the insert is manufactured of silicon, especially silicon foam. The insert can be colored especially in such a way that it is opaque, shielding the sensor head from ambient light.

The back shell and the two holding legs preferably form a holding areas system whose main pressure axes intersect at the center of the finger and extend relative to each other at angles of roughly 120°.

The material of the insert can to some degree can extend radially between each of the adjacent holding areas or gaps.

Further particulars and characteristics of the invention are clear from the following description in connection with the drawing. In the drawing:

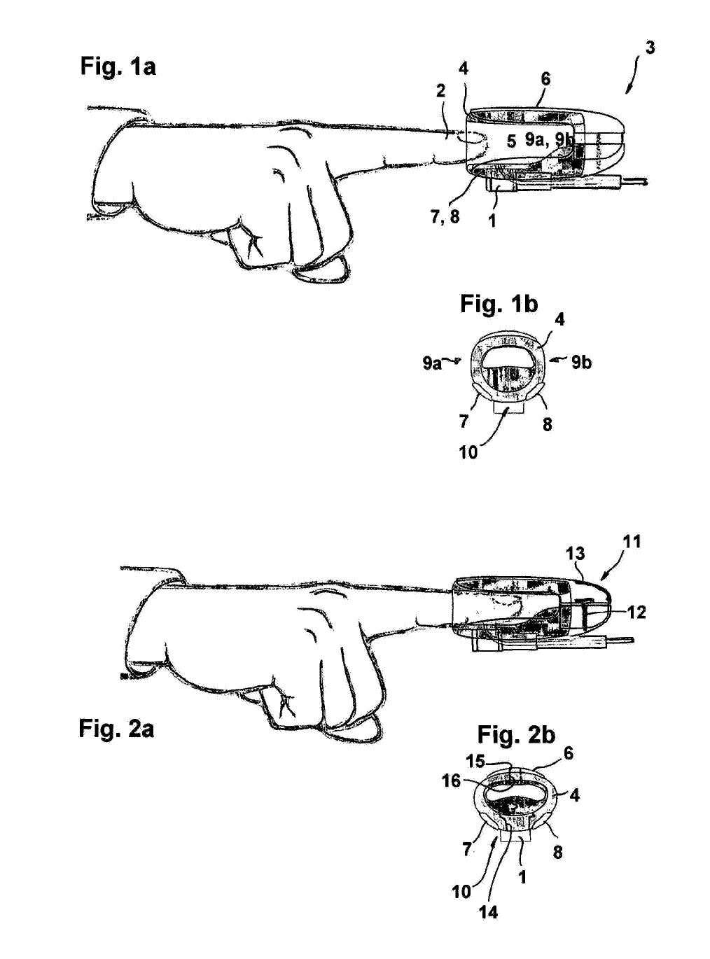

FIG. 1a is a view illustrating the structure and the use of a holder according to the invention for a sensor head on a user's finger, here when open immediately before use;

FIG. 1b is a cross section through the holder according to the invention according to FIG. 1a when relaxed;

FIG. 2a is a view illustrating the holder according to the invention in applied position, with compressed insert or is cushion;

FIG. 2b is a cross section through the holder according to the invention according to FIG. 2a when compressed;

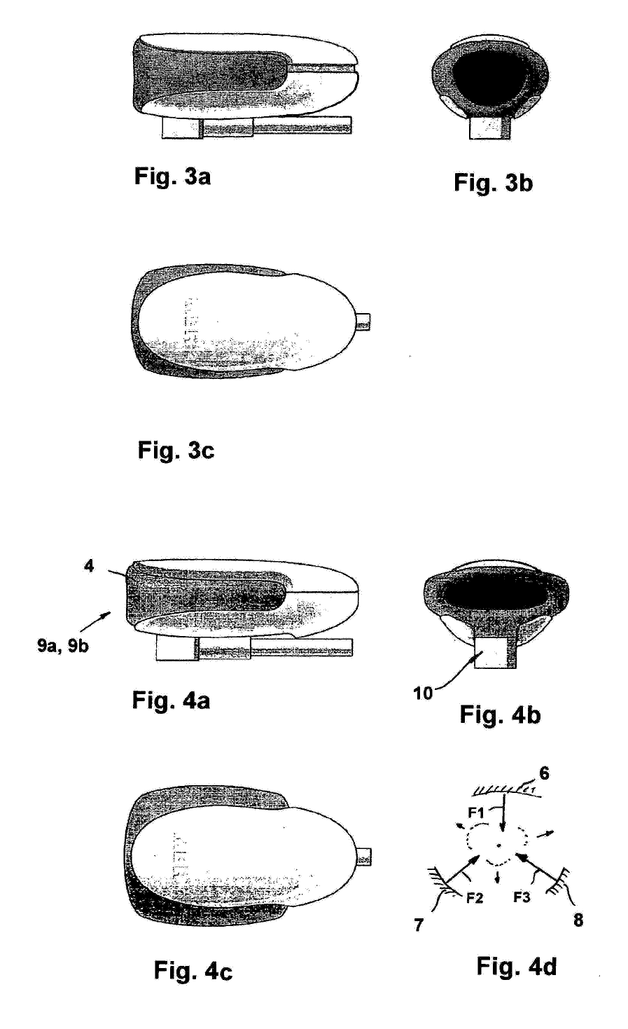

FIG. 3a is a side view of the holder according to the invention when open;

FIG. 3b another side view of the holder according to the invention when open to illustrate the shape of the cushion;

FIG. 3c is a top view on the holder according to the invention when open;

FIG. 4a is a side view of the holder according to the invention when closed;

FIG. 4b is another side view of the holder according to the invention when closed to illustrate the radial cushion bulge;

FIG. 4c is a top view on the holder according to the invention when closed;

FIG. 4d is a diagram showing the force system acting at the insert.

FIG. 1 shows a holder for applying a sensor head 1 to a user's finger 2, with an outer housing 3, an insert 4 that is made of a soft material and provided in the outer housing 3, the insert 4 forming a finger stall 5.

The outer housing 3 is formed by a back shell 6, a first holding leg 7, and a second holding leg 8. The two holding legs 7 and 8 can be positioned variably in such a way with respect to the back shell 6 that the insert 4 between the back shell 4 and the two holding legs 7 and 8 is deformed in a defined way.

As can be seen in FIG. 1b, respective holding gaps 9a and 9b are formed between the back shell 3 and the holding legs 7 and 8. The insert 4 is exposed at these holding gaps 9a and 9b. Moreover a leg gap 10 is formed also between the two holding legs 7 and 8, the insert being exposed also at this leg gap 10.

The holding gaps 9a and 9b and the leg gap 10 form spread zones through which the material of the insert 4 can extend radially outward in a defined way. This way a particularly advantageous compression of the insert 4 against the user's finger is made possible.

FIG. 2a shows the holder according to the invention when applied and closed.

In the embodiment shown here the back shell 6 and the holding legs 7 and 8 are coupled to be adjustable by a mechanism 11. In the embodiment shown here the adjusting mechanism 12 includes a set screw assembly 11. The set screw assembly 11 is covered by a cap 13, so that the adjusting mechanism cannot be actuated unintentionally.

As can be seen in FIG. 2b, the two holding legs 7 and 8 are united in a leg structure that as such compresses the insert 4 in a defined way against the inner side of the back shell. At the leg gap 10 an application opening 14 is provided in the insert 4, for mounting the sensor head 1. It is possible also at the back section 6 as well as if need be in the area coinciding with it of the insert 4 to provide an opening 15 or 16 for mounting a further or supplementary sensor head. The insert 4 is made of a silicon; especially silicon foam. The stall 5 of the insert 4 fittable over a distal phalange can have a special outer shape that to some degree offers an inner finger surface bed, and prevents removal of the holder by lightly gripping of the finger rearward of the joint, especially by means of a slight raised portion fitting back of the finger joint.

FIGS. 3a, 3b, and 3c further illustrate the structure of the holder according to the invention. These figures show the holder when open to fit over a relatively large finger, or at least to be pushed easily onto such a finger.

FIGS. 4a 4b, 4b and 4c show the holder according to the invention when closed or set very small. The outer sections of the insert 4 exposed at the gaps 9a and 9b and at the leg gap 10 can spread outward radially through these gaps.

As shown in FIG. 4d, the back section 6 and the holding legs 7 and 8 generate compressive forces F1, F2, and F3 effective along vectors extending angles of roughly 120° to one another. Spread zones formed between the legs and the back section 6 allow the material of the insert 4 to extrude radially outward.

The back section 6 forms a slightly larger purchase on the insert 4 than the holding legs 7 and 8. The back section 6 acts as a base for gentle squeezing of the user's finger covered by the insert 4 in accordance with the radial pressure forces F2 and F3 of the holding legs 7 and 8.

In the insert 4 the properties and thickness of the wall as well as the shape of the finger stall influence the surface pressure distribution. The insert can be also made of several materials of different Shore hardnesses. For example the area of the insert against the back of the finger may have a slightly higher Shore hardness than the area pressing against the inner finger face. The inner surface of the insert can have a shape corresponding to that of a typical finger.

The insert 4 and the outer housing formed by the back section 6 and the holding legs 7 and 8 are of complementary shape so that the insert 4 can be inserted into the outer housing while elastically deformed and snapped into it.

The sensor in a typical application form of the holder according to the invention is designed as an optical sensor head that incorporates several light guides that direct light into the finger, and light emitted by the finger can be collected and fed to a spectrometer.

The holder can be moreover provided with structures that allow it to be secured to a person's hand. For example the outer housing can be formed with a strap loop through which a strap can be passed that can be secured for example to a bracelet fitted around the user's wrist.

It is possible to form the outer housing with a small casing holding an electronic circuit so that signals detected by the sensor head can be transmitted wirelessly to a receiver.

It is possible for a given outer housing to work with different inserts, for example a set of them, so that by matching the outer housing with selected inserts of the set specific applications can be accommodated particularly advantageously.

The sensor head 1 is supported by the holder preferably in such a way that the sensor head 1 rests on the user's inner finger surface with a surface pressure that corresponds more or less to the surface pressure generated by the insert 4, or only slightly higher. The sensor head 1 can be pivoted on the insert to some degree, or gimbaled, so, depending on the elasticity of the surrounding area of the insert, it automatically occupies a favorable orientation. The outer housing provides a strain relief for the sensor head so that possible traction forces acting on a wire extending away from the holder do not act directly on the sensor head 1, but are absorbed by the outer housing. This ensures that possible forces acting on the sensor cable do not falsify the measurement e.g. by temporarily moving the body tissue out of contact with the sensor head.

According to a particularly preferred embodiment of the invention the holder is configured such that the sensor head can be detached from the holder and fitted to it even when the holder is mounted on the user. In this way it is possible to separate the sensor head rapidly from the user and to attach it later again in the same position. The gripping of the sensor head 1 can be done by the holding legs 7 and 8. The holding legs 7 and 8 can form a detent so the sensor head 1 is on the one hand sufficiently secured and placed, and on the other hand is still sufficiently mobile in order to occupy an optimal orientation on the corresponding finger area.

The outer housing 1 preferably forms a shell-like body of oval shape. The outer housing includes a closed egg-shaped upper part to which the back section 6 and the two holding legs connect as slightly more elongated structures. The back section 6 and the holding legs are separated from each other by the holding and leg gaps. These gaps form windows where the insert incorporated in the outer housing 1 is partially exposed. Gripping force for the insert is set by adjusting the distance between the inner surfaces of the back section 6 and of the holding legs 7 and 8. The part forming the back section 6 of the outer housing 1 can have a sectional shape forming a linear guide between the part forming the back section 6 and the part forming the holding legs 7 and 8. This linear guiding is preferably such that the back section 6 and the holding legs 7 and 8 are adjustable with respect to each other along an axis that is substantially perpendicular to the longitudinal axis of the finger stall and also substantially perpendicular to the longitudinal axis of the back section 6.

1. A holder for applying a sensor head to a user's finger, the holder comprising:
an outer housing, and
an insert made of a soft material, in the outer housing, and forming a finger stall, the outer housing including a back shell, a first holding leg and a second holding leg, the two holding legs being variably positionable with respect to the back shell in such a way that the insert between the back shell and the two holding legs is deformed in a defined manner.

2. The holder according to claim 1, wherein a holding gap is formed between the back shell and the holding legs and the insert is exposed at this holding gap.

3. The holder according to claim 1, wherein the two holding legs form a leg gap, the insert is exposed at this leg gap, and the insert can spread radially into this leg gap.

4. The holder according to claim 1, wherein the back shell and the holding legs are relatively displaceable by an adjusting mechanism.

5. The holder according to claim 4, wherein the adjusting mechanism includes a set screw assembly.

6. The measuring device according to claim 5, wherein a cap is provided for covering the set screw assembly.

7. The measuring device according to claim 1, wherein the two holding legs are formed as a one-piece leg structure.

8. The measuring device according to claim 1, wherein an application opening is provided at the leg gap for mounting a sensor head.

9. The measuring device according to claim 8, wherein another application opening is provided at the back section for holding another sensor head.

10. The measuring device according to claim 1, wherein the insert is made of silicon.

11. The measuring device according to claim 10, wherein the insert is made of silicon foam.

12. The measuring device according to claim 1, wherein the insert is made of a substantially opaque material.
